(19) United States
(12) Patent Application Publication
(10) Pub. No.: US 2007/0175484 A1
(43) Pub. Date: Aug. 2, 2007

(54) Condoms for beneficial agents delivery

(76) Inventor: Robert J. Staab, Allendale, NJ (US)

Correspondence Address: Evelyn M. Sommer, Suite 825, 250 Park Avenue, New York, NY, 10022, US

(21) Appl. No.: 11/341,960

(22) Filed: Jan. 30, 2006

(51) Int. Cl. A61F 6/04

(52) U.S. Cl. 128/844

(57) ABSTRACT

An improved condom is provided by coating the inner and or outer surface of the condom base with a dissolvable element containing an agent material for delivering the agent material during sexual intercourse. The dissolvable element is made of dissolvable polymer material and/or complex carbohydrate material which are food grade materials and have selected dissolution properties, such that it remains in substantially solid form before use, and dissolves due to human body temperatures and moisture during use to release the agent material in a desired timed release and dosage. The dissolvable element is preferably a film made of polyvinyl alcohol, polyethylene oxide, hydroxypropyl methyl cellulose and/or carboxymethyl cellulose. The dissolvable element may be formed as a single layer of film or a laminate of different film layers for compound release properties. The dissolvable element is at least partially foamed using inert gases, such as nitrogen, to adjust its dissolution properties. The agent material can be selected from the group consisting of spermicides, anti-infectives, anti-inflammatories, antibodies, steroids, vasoactive agents, sensory modifying agents, anti-oxidants, preservatives, plasticizers and mixtures thereof. The condoms can be used for sexual intercourse involving penis with vagina, rectum or mouth.

### FIELD OF THE INVENTION

This invention relates to an improved condom and more particularly to a condom with beneficial formulations coated on its inside and/or outside surface which can be dissolved in a controlled manner during sexual intercourse to provide sensory, medical and other benefits.

### BACKGROUND FOR THE INVENTION

It is well known that condoms can be used during sexual intercourse to prevent pregnancy and significantly reduce the risk of sexually transmitted diseases. Of particular relevance to the present invention are coated condoms which possess actives in the coating for medical and recreational purposes, such as for further reducing the likelihood of transmitting sexually transmitted diseases, contraception, and treating male impotence.

U.S. Pat. Nos. 4,829,991, 6,056,966 teaches the application of the pharmaceutical compositions effective in treating male impotence and erectile dysfunction through topical administration on the inside surface of the condom for treating these conditions. Such mode of administration serves the dual purposes of promoting erection and providing a contraceptive and disease-transmission barrier during intercourse.

U.S. Pat. No. 4,898,184 teaches a condom with spermicide applied to the inside and/or outside surface for contraceptive purposes.

U.S. Pat. No. 5,878,747 teaches coating the inner and/or outer surface of condoms with acidic polysaccharides for reducing the risk of HIV infection.

While the prior art discloses condoms coated with various agents for providing medical and recreational benefits, the coating material and coating process can be significantly improved as by allowing for more tunable and controlled release of the actives.

The present inventor has previously been granted two US utility patents (U.S. Pat. Nos. 5,529,782 and 5,393,528) on devices adapted for local administration of an agent material in an internal body cavity, the entireties of which are incorporated herein by reference thereto. The device comprises a dissolvable element and an agent material carried in such dissolvable element. During use, the heat and humidity at the internal body cavity, such as the vagina, rectum, or mouth, in addition to the friction produced in use, dissolves the dissolvable element and releases the agent. The dissolution properties of the dissolvable element can be readily tuned and controlled, at least partially, by adding nitrogen or other suitable gases in forming a film of the dissolvable element.

It has occurred to the present inventor that an improved condom can be produced by applying the inert gas-treated dissolvable element with improved dissolution properties, which carries at least one kind of desired agent material, to the inner and/or outer surface of the condom. The dissolvable element remains in substantially solid form before use, and dissolves primarily due to human body temperatures and moisture during use to release the agent material in a desired timed release and dosage.

### SUMMARY OF THE INVENTION

In accordance with the present invention, an improved condom adapted for delivery of beneficial agents, comprise a substantially tubular body of flexible film having an open end, a closed end, an outer surface, an inner surface, and at least one coating of an inert gas-treated dissolvable element containing at least one type of beneficial agent, applied to its outer and/or inner surface. The tubular body is formed of a thin homogeneous elastomeric membrane having a generally constant diameter from its open to its closed end to thereby define a longitudinally extending chamber. The surface of the tubular body or condom can be smooth, ribbed or of any texture known in the art. The thin homogeneous elastomeric membrane may be made of latex, natural caecum or other material of the type used in conventional condoms. The condom of the present invention includes at least one coating of a dissolvable element applied to the outer and/or inner surface of the tubular body in the form of a film. The film can be of a single layer or a laminate of more than one layer of similar or dissimilar elements. The dissolvable element further carries at least one beneficial agent such as contraceptive, anti-infective such as antiviral, antibacterial and anti-fungal agents, penile erection stimulant such as vasodilator agents, sensory modifying agents, plasticizer such as glycols, glycerin and water for improving the lubricity and softness, etc. The dissolvable element is treated with a gas, preferably an inert gas such as nitrogen, for improved dissolution properties. The condom can be of any known type, such as those adapted for use by the male partner or female partner. During use, the condom is applied onto the penis of the male partner or into the vagina of the female partner, and the heat and moisture naturally present in the body cause the dissolution of the dissolvable element and thereby releases the beneficial agents.

### DESCRIPTION OF THE DRAWINGS

FIG. 1 is a side view of an improved condom in accordance with the invention.

FIG. 2 is a sectional view taken on the line 2-2 of FIG. 1 in accordance with a first embodiment.

FIG. 3 is a sectional view taken on the line 3-3 of FIG. 2.

FIG. 4 is a sectional view taken on the line 2-2 of FIG. 1 in accordance with a second embodiment.

FIG. 5 is a sectional view taken on the line 2-2 of FIG. 1 in accordance with a third embodiment.

FIG. 6 is a sectional view taken on the line 2-2 of FIG. 1 in accordance with a fourth embodiment.

### DETAILED DESCRIPTION OF THE INVENTION

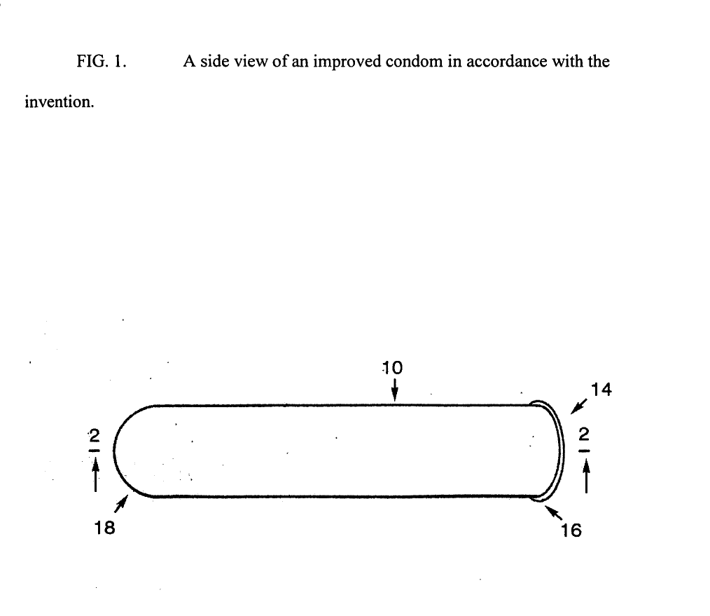

Condoms in accordance with the invention are illustrated in FIGS. 1-6. As illustrated in FIG. 1, the condom comprises a tubular body 10 with an open end 14 and a closed end 18. The tubular body 10 and the closed end 18 both have an inner surface and an outer surface. The inner and/or outer surface can be smooth, ribbed or any texture known in the art such as those disclosed in U.S. Pat. Nos. 3,809,090 (having rigid projections on the outer surface) and 4,852,586 (having a plurality of antipodal pairs of projections extending through the barrier membrane). The open end of the condom further comprises a rim 16.

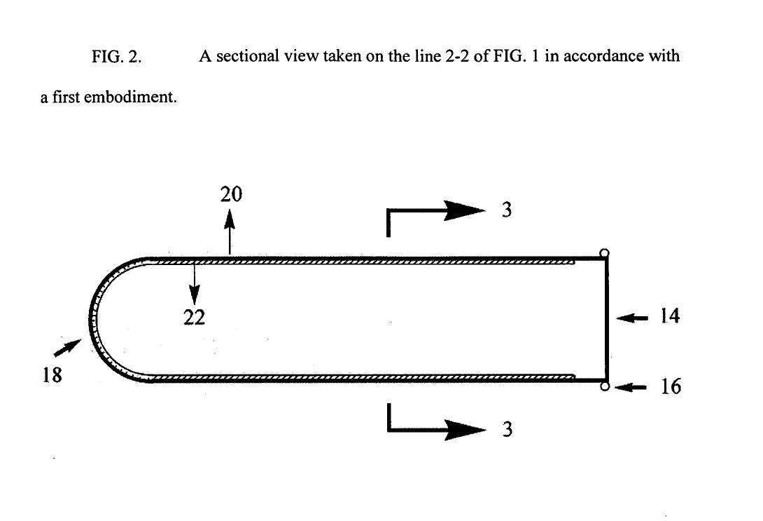

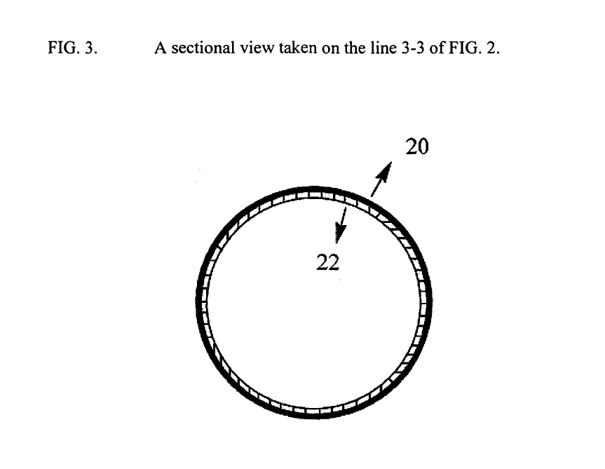

A first embodiment of the invention, adapted for delivering the beneficial agent to the penis, is shown in FIGS. 2 and 3. The tubular body is shown to comprise at least two layers of material. The outer layer, 20, the base of the condom in this embodiment, is a thin homogeneous elastomeric and prophylatically impermeable material such as latex, rubber or other materials such as natural sheep caecum which is generally used in the prior art to form the body of a conventional condom. The inner layer, 22, is a coating of a dissolvable element carrying at least one type of beneficial agent. The coating can be applied as a thin film throughout the inner surface of the condom or part of the inner surface. The coating can also be applied in a patterned manner, such as in the form of isolated islands. Preferably the inner surface of the condom is uniformly coated except the section that is about 25 mm from the open end. The coating is preferably about 3 micrometer thick and has no appreciable impact on the modulus, elasticity, prophylaxis, contraceptive or other benefits of standard condoms. It is also conceivable to have more than one layer of coatings in the form of a laminate or a mixture of dissolvable elements on the inside surface of the condom and each layer may comprise one dissolvable element with a unique dissolution property and at least one beneficial agent with different concentrations, distributions and/or functions. When the outer layer comprises latex or rubber, the condom can be manufactured using any of the known processes. For example, the condom may be formed by injection molding or dipping to produce the outer layer with the coating added by an additional dipping, molding or rinse step prior to packaging. A similar post-manufacturing step would be used for natural caecum condoms.

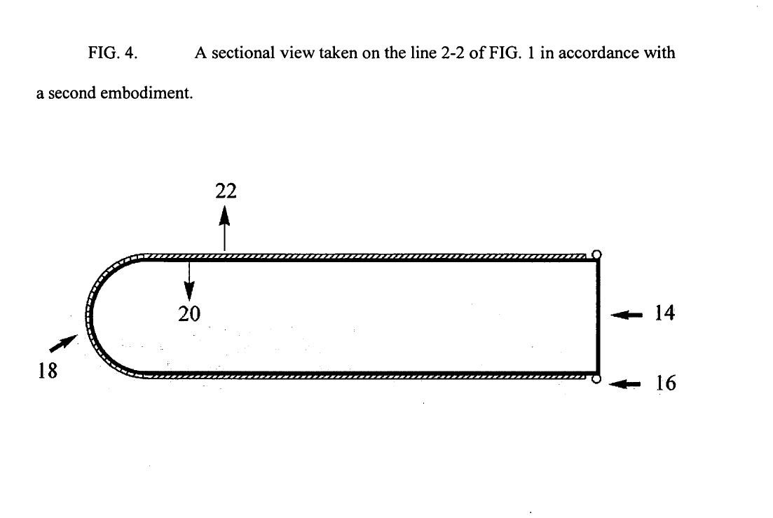

A second embodiment of the present invention is illustrated in FIG. 4. The condom in this embodiment is adapted for delivering the beneficial agent to the vagina. The condom is constructed in a similar manner as the first described embodiment except that the coating 22 is on the outer surface of the condom. Multiple coatings can be applied using similar or dissimilar dissolvable elements and beneficial agents. Materials and manufacturing processes similar to that for the first embodiment can be employed.

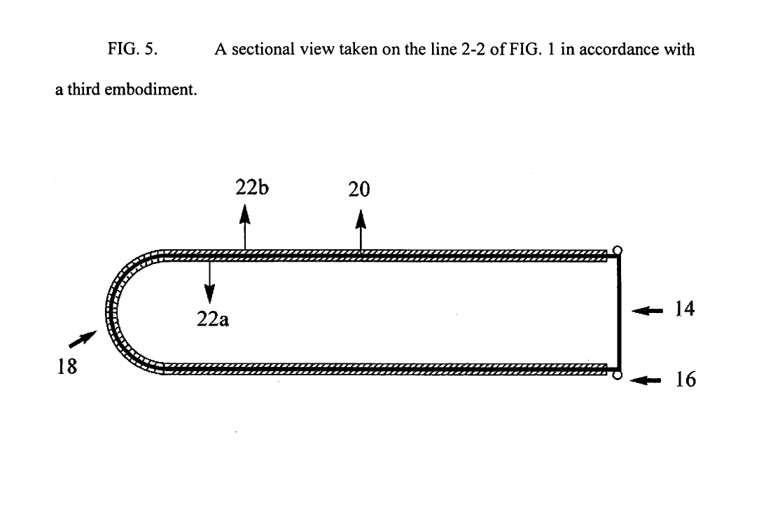

A third embodiment of the present invention is illustrated in FIG. 5. The condom in this embodiment is adapted for delivering the beneficial agent to both the penis and vagina. Both the inner and outer surfaces of the condom are coated with similar or dissimilar dissolvable elements (22a and 22b) incorporating the same or different beneficial agents.

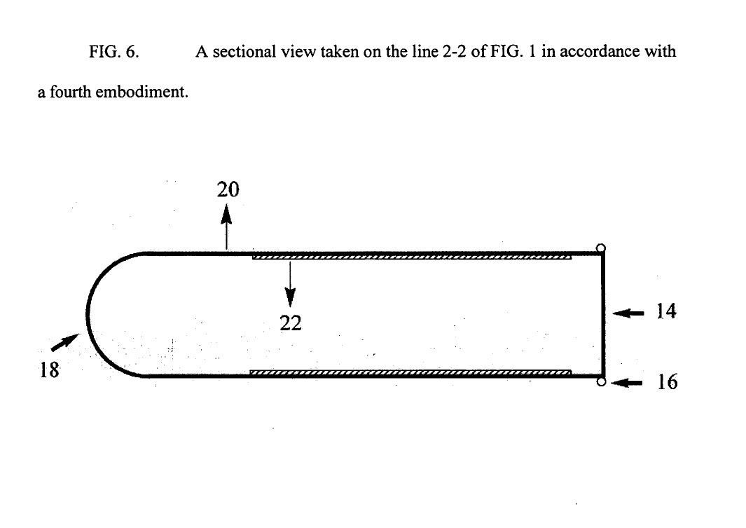

A fourth embodiment of the present invention is illustrated in FIG. 6, which is a variant based on the first embodiment. Instead of having a coating essentially throughout the inner surface of the condom, the distal portion of the inner surface, including the inner surface of the closed end and optionally a section of the inner surface of the tubular body adjacent the closed end, is not coated with the dissolvable element and beneficial agent. Similarly, the second and third embodiments can be varied to exclude part of the surface from being coated.

The dissolvable element can be of any nontoxic material that is stable in a solid form during storage, is compatible with the beneficial agent incorporated therein, and can be dissolved to release the beneficial agent during use due to the body heat and moisture and possibly further aided by friction. The dissolvable material is preferably a food-grade material safe for internal use. The dissolvable element is designed to be stable at typical temperatures (e.g. up to 140° F.) and humidity (e.g. up to 99% relative humidity) encountered during storage so as to remain in substantially solid form and not begin dissolving prior to the intended use. Lubricity is another desirable property for use in the vagina and other internal areas where sensitive tissues are likely to be encountered. A preferred dissolvable element is in the form of a film and is made of a polymeric material, such as polyvinyl alcohol, polyethylene oxide, and/or complex carbohydrate material or combinations thereof.

The preferred dissolvable element is in the form of a film made of the combination of suitable grades polyvinyl alcohol, polyethylene oxide, and/or complex carbohydrate material. Polyvinyl alcohol (PVA) is a preferred material for the film because it is non-toxic and medically safe to use internally. PVA comes in different grades that can be classified as cold water soluble (dissolves from 40 to 212° F.), intermediate soluble, dissolving (110 to 212° F.), fully hydrolyzed (140 to 212° F.), and superhydrolyzed (180 to 212° F.). PVA is commercially available from companies such as Air Products Company, of Allentown, Pa. The cold water soluble and intermediate dissolving grades are the most useful for the desired moisture and heat-dissolving properties required for contraceptive purposes. A particularly preferred cold water soluble grade of PVA is an 80% hydrolyzed polyvinyl alcohol having a molecular weight of 9,000-10,000; for intermediate solubility, an 87-89% hydrolyzed polyvinyl alcohol having a molecular weight of 13,000-23,000 for a slow dissolving, a 98-99% hydrolyzed polyvinyl alcohol having a molecular weight of 31,000-50,000 and for the least dissolving, a fully hydrolyzed >99% of polyvinyl alcohol having a molecular weight of 85,000-186,000 being preferred. All of the aforementioned polyvinyl alcohol preparations are available from Aldrich Chemical, Milwaukee, Wis. However, in the invention, a film of the higher temperature or water soluble grade may be combined with a film of the lower temperature or water soluble grade in order to alter the temperature dissolution and moisture, solubility and stability properties so that the film can be used most suitably in the vaginal environment. The PVA material or materials selected for contraceptive, anti-infective or penile erection stimulating uses typically dissolve relatively quickly, e.g., over several minutes, or in some cases as quickly as in several seconds.

Polyethylene oxide is another satisfactory material for forming the film because it has very good moisture, particularly humidity, stability and further is a food contact grade material. It is very compatible with the spermicide nonoxynol-9 and many other medications. It also has the added benefit of good lubricity, which makes the film structure even more comfortable to insert and use during sexual activity. Preferred polyethylene oxide materials are sold by Union Carbide Corp., of Danbury, Conn., in molecular weights of from 50,000 to 8,000,000 Daltons. The polyethylene oxide is available from Aldrich Chemical, Milwaukee, Wis.

A complex carbohydrate material suitable for use in the film is hydroxypropyl methyl cellulose, or carboxy methyl cellulose which is sold, for example, under the trademark “Methocel” by Van Waters & Rogers, Inc., of Seattle, Wash. This material is also food-grade, medically safe to use internally, low cost, and very stable in a humid environment. “Methocel” is cellulosic in nature being derived from trees. It is dissolvable in the same temperature ranges as PVA. Hydroxypropyl methyl cellulose is a particularly preferred material for use in forming the films of the invention. Its acceptance by the FDA as a direct food additive is well known (CAS 9004-65-3). The preferred hydroxypropyl methyl cellulose has an average molecular weight of about 86,000.

The dissolvable element, preferably in the form of a film, may be a laminate of two or more layers of different polymer materials, or may be a single layer with two or more polymer ingredients mixed together. Further, the film laminate may be a gas foamed film or constructed of layers of different gas foamed films or of layers of both non-foamed and gas foamed films. The exact mixture used will depend upon the intended use and combination of properties and/or effects desired, which may include heat-dissolving temperature range, time release period, lubricity, shelf life, turgidity, stability in a moisture environment, compatibility with spermicides and/or medications etc. In the case of contraception, two films may be used which dissolve at varying rates. Such a laminate device can offer prompt efficacy upon insertion combined with extended contraceptive protection with dissolution taking place over a period of many hours. Thus in accordance with the invention, the film may be constructed as a laminate composed of gas foamed film with non-gas foamed film layers, polyvinyl alcohol and polyethylene oxide film layers, polyvinyl alcohol and hydroxypropyl methyl cellulose layers, in all possible combinations. The laminates can be formed in the conventional manner, for example the mixture in liquid form will be poured or cast on to a plate or into a mold and allowed to begin to set, at which time another liquid mixture of different composition will be poured on to the first setting up mixture, and both mixtures allowed to set up completely producing a laminate or layers of different materials. Further, the laminate can be applied to the condom as final stage dipping processes.

Fully formed films can also be laminated to each other through use of an adhesive. A preferred adhesive is a dilute aqueous solution of the polymer from which the film has been made. Thus, for example, a polyvinyl alcohol film could be adhered to another polyvinyl alcohol film through use of a dilute solution of polyvinyl alcohol.

The agent material to be administered locally may be spermicide for contraceptive use, and/or drug or medication. The agent material is evenly distributed throughout the film, so that as the film slowly dissolves, it releases the agent material in the proper dosage to perform its spermicidal or medicating function. The agent material is selected for compatibility with the polymer material and its dissolution characteristics. The device of the invention thus is composed of a biologically compatible material that has been blended homogeneously with a spermicide or drug which is released into a body cavity at a controlled rate upon contact with the body fluid.

Some spermicides have good surfactant properties which facilitate dissolving and dispersing in vaginal fluids. Examples of those suitable for use include nonylphenoxypolyethoxy ethanol (sold under the trademark “Nonoxynol-9”), p-methanyl phenylpolyoxyethylene ether (Menfegol), polyoxyethylene oxypropylene stearate, polyoxethylene laureate, glycerol ricinolate, mono-iso-octyl phenyl ether, polyethylene glycol, methoxy polyoxyethylene glycol 500 laureate, polyoxyethylene stearylamine, benzalkonium chloride, cetyl trimethylammonium bromide, methyl benzethonium chloride, sodium dodecylsulfate, nonylphenol polyethylene sodium sulfate, sodium oleate, zinc phenosulfonate, dodecyl diaminoethyl glycine, p-diisobutylphenoxy polyethanol (Octoxynol), dodecamethylene glycol monolaureate, sodium lauryl sulfate and the like. A suitable dosage of nonoxynol-9 for contraceptive use is about 100 to 150 mg. However, the amount of spermicide may vary in accordance with their rate of release from the device and the spermicidal efficacy.

The dissolvable element may be used to deliver a medication internally in the vaginal or cervical area in combination with a spermicide or alone. Suitable medications which can be delivered with the film include: (1) anti-infectives such as antibiotics, sulfonamides, antivirals, antifungals, antiprotozoan and antibacterials; (2) anti-inflammatories, such as hydrocortisone, dexamethasone, triamcinolone, and various prednisolone compounds; (3) estrogenic steroids, such as estrone; (4) progestational agents, such as progesterone; (5) prostaglandins; (6) coronary vasodilators; (7) sensory modifying agents. Monoclonal antibodies such as those useful against cell surface components or against pathogenic organisms such as the human-immuno-deficiency (HIV) family of viruses may be incorporated into the device of the present invention for ultimate intravaginal release. Sensory modifying agents include the agents which can reduce penile sensation and thereby prolong sexual intercourse. Combinations of the various drugs may be used as desired. Typically the range of drug additives may be in the amount of 0.0001% to about 50% by weight. The medications may be in a variety of chemical forms, such as uncharged molecules, molecular complexes, or nonirritating, pharmacologically acceptable salts. Simple derivatives of such medications, such as ethers, esters, amides, and the like, can also be used for desirable properties such as retention, release, and easy hydrolyzation by body pH, enzymes, etc. The amount of medication to be used varies depending upon the particular drug, the desired therapeutic or prophylactic effect, and required release times.

The dissolvable element may also include plasticizer material, such as water, glycols, glycerin, and like materials, in order to enhance lubricity and softness. While water is suitable as a plasticizer it is not useful in all cases, but this factor can be readily ascertained. A preferred plasticizer is glycerin USP, sold by Van Waters & Rogers, Inc., in either natural or synthetic form. Glycerine (glycerol) CAS 56-81-5 is particularly preferred. The plasticizer may be added in any desired concentration, for example, from 0.1% to 35%, for better handling and lubricity. The softness and flexibility of the dissolvable film, due to its thin layer structure without any rigid elements, and particularly when combined with plasticizer, ensures that the device may be worn with complete comfort and will not be felt by the sexual partners.

Various preservatives, antifungal agents, antibacterial agents, antiviral agents, antiprotozoal agents, and antioxidants may also be added if desired. Flavors, fragrances, and/or coloring agents may be incorporated into the film. The polymer film may be substantially transparent, or may be embossed with indicia or colored with opaquing agents. These additives may be present in any desired concentration, for example, from 0.001% to 50%. The concentrations of these additives will depend upon the desired properties, the agent to be released, the potency, the desired dosage, dissolution times, etc. In preparation, the polymer solids, water, or other solvent, contraceptive and/or medicinal, glycerine etc. are admixed in the proper concentrations and the mixture heated to the appropriate temperature for dissolution and for formation of a uniform blend to take place. The heating can take place, for example, by submerging vessels containing the mixture in water or jacketed vessels held at constant temperature, for example 104-140° F. The mixture can either be cast directly or transferred into another water bath of cooler temperature, for example 68-104° F., and other heat sensitive ingredients introduced with stirring. The application of heat is, however, not necessary, which is advantageous when pharmaceuticals or other agents to be added are heat sensitive.

Several formulations utilizing different polymers as well as different active ingredients are listed below:

I. Benzalkonium Film, 19 mg benzalkonium chloride

| | |
|---|---|
| hydroxypropyl methyl cellulose | 52.5% |
| glycerine | 37.5% |
| BZK* | 10.0% |
| | 100.0% |
| nitrogen gas added to form foamed web | <1.0% |

*BZK, e.g., BTC-50, a 50% aqueous active drug from Stepan
Company, Northfield, Illinois.
Chemical, Jersey City, NJ

II. Nonoxynol-9 Film, 100 mg nonoxynol-9

| | |
|---|---|
| hydroxypropyl methyl cellulose | 36.0% |
| glycerine | 29.0% |
| nonoxynol-9* | 35.0% |
| | 100.0% |
| nitrogen gas added to form foamed web | |

*nonoxynol-9 from Rhone Poulenc, NJ

III. Clotrimazole Film, 100 mg clotrimazole

| | |
|---|---|
| hydroxpropyl methyl cellulose | 36.0% |
| glycerine | 29.0% |
| clotrimazole* | 35.0% |
| | 100.0% |
| nitrogen gas added to form foamed web | |

*clotrimazole from e.g., Flavine, Inc. Closter, NJ

IV. Vaginal Deodorant Film

| | |
|---|---|
| polyethylene oxide | 55.0% |
| glycerine | 35.0% |
| fragrance | 10.0% |
| | 100.0% |
| nitrogen gas added to form foamed web | |
| fragrance from e.g., IFF, Dayton, NJ | |

V. Vaginal Lubricant

| | |
|---|---|
| polyethylene oxide | 75.0% |
| glycerine | 25.0% |
| | 100.0% |
| nitrogen gas added to form foamed web | |

The film characteristics are altered by adding appropriate amounts of gas, such as air, nitrogen, or other inert gases, which can produce a more acceptable film texture and modify the dissolution rates accordingly. For example, it has surprisingly been found that the addition of nitrogen or other inert gas to a PVA film containing nonoxynol-9 halves the dissolution rate of the film. The fine tuning of dissolution rates and delivery of agent material, by the addition of gases and by altering the grades or mixtures of polymer materials or layers, is an important aspect of the present invention.

On addition of the gas, preferably nitrogen, a web is formed of the final formulation and the gas. The resultant structure can be described as a foam with various sized air bubbles trapped in the matrix. There is a dual benefit that has been surprisingly observed in this connection, namely that not only can the size of the bubbles in the foam alter the dissolution rates and correct what is a serious flaw in standard polymer films, it also offers to the user a perceptible softness to the film which enables the delivery of many types of drugs to tender mucosal tissues. It has been observed that the formation of this web of the polymer/drug formulation and the gas must be made just prior to casting on the glass or steel plates. This offers precise control over the microbubbles and resultant control over the dissolution.

Without this web formation, the quick release of drug was heretofore not possible. This frothy foam mixture or web can also be added to a mold to provide a formed device such as a barrier delivery system which completely dissolves upon use in a body cavity, e.g. the vagina.

The gases, for example, air or nitrogen are introduced near the point of application of the liquid polymer material to the stainless steel casting sheet. The gases are added in a closed system by mixing with whipping blades or a motor driven homogenizer to homogenize the mixture of polymer, active material and gas to form a frothy foam.

The film coated on the condoms is intended to remain in solid form, for shelf storage or prior to use, at temperatures up to 140° F. Controlled environmental storage (temperature and humidity) of the film prior to use may be desirable if ambient temperatures and humidity are extreme. Once the condom is in contact with the vaginal tract, the film will dissolve due to the moisture and body temperature in the vagina, typically 98.6° F. As the spermicide or drug is released from the dissolving film, it becomes mixed in the vaginal fluids and adheres to the tissues where it forms an effective matrix for drug action, e.g., spermicidal or anti-infective action. After use, the condom is removed manually and any drug remaining in the vaginal fluids are both removed naturally from the body by washing.

When more than one layer of coating is applied or the coating is a laminate of more than one type of films, one of the layers of the coating or the film can be made of slower dissolving polymer material for release of more drug or another drug material in combination, for example, a spermicide and an anti-infective or anti-inflammatory medication. A third (and additional) layer(s) with additional drug can also be employed for sustained release of the drug. Laminate of foamed or combinations of foamed with non-foamed layers are used to readily control the dissolution rate of the coating. Thus, a composite of desired release properties and agent materials is obtained.

I claim:

1. A condom capable of delivering at least one agent material into a body cavity characterized by improved heat and humidity stability of up to 140° F. and 97% respectively, comprising a condom base having an inner and outer surface coated on at least one surface thereof with at least one dissolvable film from a member selected from the group consisting of polyvinyl alcohol, polyethylene oxide, hydroxyl propyl methyl cellulose and mixtures thereof, at least one of said films having distributed throughout an inert gas, at least one of said films incorporating at least one agent material, said film dissolving at the temperature of said body cavity and in the presence of the moisture naturally present in the said body cavity to release said agent material.

2. A condom according to claim 1 wherein said condom base is formed of a material selected from the group consisting of rubber, latex and caecum.

3. A condom according to claim 1 wherein said agent material is a member selected from the group consisting of spermicides, anti-infectives, anti-inflammatories, antibodies, steroids, vasoactive agents, sensory modifying agents, anti-oxidants, preservatives, plasticizers and mixtures thereof.

4. A condom according to claim 1 wherein said film is a laminate formed of at least two film layers, each of said layers comprising a group member characterized by different dissolution properties.

5. A condom according to claim 1 wherein said internal body cavity is selected from vagina, rectum and mouth.

6. A condom according to claim 1 wherein the dissolution rate of at least one of the said films is adjusted by varying the amount of inert gas introduced into said film.

7. A condom according to claim 1 wherein said inert gas is nitrogen.

8. A condom according to claim 3 wherein said plasticizer is selected from the group consisting of glycols, glycerin, water and mixtures thereof.

9. A condom according to claim 1 coated on said inner and outer surfaces with said at least one dissolvable film.

10. A condom according to claim 1 coated on said inner surface with said at least one dissolvable film.

11. A condom according to claim 1 wherein said inner surface is only partially coated with said at least one dissolvable film.

12. A condom according to claim 1 wherein said coating comprises hydroxyl propyl methyl cellulose, glycerine and BZK.

13. A condom according to claim 1 wherein said coating comprises nonoxynol-9, hydroxyl propyl methyl cellulose and glycerine.

14. A condom according to claim 1 wherein said coating comprises hydroxyl propyl methyl cellulose, glycerine and clotrimazole.

15. A condom according to claim 1 wherein said coating comprises polyethylene oxide, glycerine and a fragrance.

16. A method of manufacturing a condom having an interior surface and an exterior surface which comprises coating at least one of said surfaces with a coating comprising a dissolvable film formed from a member selected from the group consisting of polyvinyl alcohol, polyethylene oxide, hydroxyl propyl methyl cellulose and mixtures thereof incorporating at least one active agent material selected from the group consisting of spermicides, anti-infectives, antibodies, steroids, vasoactive agents and mixtures thereof.
